(19) United States
(12) Patent Application Publication
(10) Pub. No.: US 2023/0124656 A1
(43) Pub. Date: Apr. 20, 2023

(54) An Antiperspirant Composition

(71) Applicant: Conopco, Inc., d/b/a UNILEVER, Englewood Cliffs, NJ (US)

(72) Inventors: Shahin ALLAHBASH, Chennai (IN); Sameer Keshav BARNE, Bangalore (IN)

(73) Assignee: Conopco, Inc., d/b/a UNILEVER, Englewood Cliffs, NJ (US)

(21) Appl. No.: 17/922,758

(22) PCT Filed: May 3, 2021
(86) PCT No.: PCT/EP2021/061539

§ 371 Date: Nov. 1, 2022

(30) Foreign Application Priority Data
May 7, 2020 (IN) 202021019492
Jun. 30, 2020 (EP) 20183171.6

(51) Int. Cl. A61K 8/73; A61Q 15/00; A61K 8/89; A61K 8/34; A61K 8/86

(52) U.S. Cl. CPC A61K 8/731; A61Q 15/00; A61K 8/89; A61K 8/34; A61K 8/86

(57) ABSTRACT

The present invention is in the field of personal care compositions, in particular compositions comprising antiperspirant actives. The present invention more particularly relates to compositions which do not contain the conventional and well established metal based actives, yet exhibit similar efficacy. This is achieved through use of a combination of a film forming polymer, a specific solvent and a particulate filler.

### FIELD OF THE INVENTION

The present invention is in the field of compositions comprising antiperspirant actives, particularly cosmetic compositions. The present invention more particularly relates to compositions which do not contain metal based antiperspirant actives yet exhibit efficacy similar to or better than that of well known aluminium based actives.

### BACKGROUND OF THE INVENTION

The present invention relates to compositions, such as those that contain antiperspirant actives. These actives are added to compositions to reduce perspiration on application to the surface of the body, particularly to the underarm regions of the human body viz. the axilla. Antiperspirant actives are typically astringent metal salts such as those of aluminium or zirconium salts. Antiperspirant actives are usually incorporated in compositions at low pH, in the range of 2 to 7. There has been a thrust to develop anti perspirant actives which are less astringent and the approach has been to look for actives that do not contain metal like aluminium. The present inventors with their extensive research in the field of such actives have hit upon a formulation which is a combination of a specific film forming polymer of modified cellulose, an organic solvent and a particulate filler which is found to have similar or better efficacy as compared to that of well-established aluminium based actives.

Certain antiperspirant compositions having low or no amount of metal actives been reported e.g. EP0440387A1 (Unilever, 1991) discloses an antiperspirant composition which is suitable for topical application to human skin, comprising: (i) from 4 to 95 percent by weight of hydrophobically-treated clay; and (ii) from 96 to 5 percent by weight of a carrier medium comprising an alkanol having from 1 to 4 carbon atoms; the composition containing not more than 0.2 percent by weight of aluminium.

FR3014314 (L'Oreal, 2013) discloses a cosmetic composition, comprising a cosmetically acceptable medium, containing a fatty phase and an aqueous phase, said aqueous phase comprising one or more monoalcohol (s) comprising from 2 to 8 carbon atoms and said fatty phase comprising at least one lipophilic film-forming polymer said lipophilic film-forming polymer being neither a vinyl polymer having at least one unit derived from carbosiloxane dendrimer, nor a silicone resin MQ, wherein the amount of monoalcohol (s) is from 8% to 40% by weight relative to the total weight of said composition.

While antiperspirant compositions devoid of metal based actives are known and used, it is desirable to develop compositions that provide better and better efficacy. It is thus an object of the present invention to provide for an antiperspirant composition which exhibits high antiperspirant efficacy while having reduced astringency.

### SUMMARY OF THE INVENTION

The first aspect of the invention relates to an anti-perspirant composition comprising:

(i) a film forming polymer selected from modified cellulose having a solubility in water of less than 1 wt %;

(ii) an organic solvent having a Hansen Solubility Parameter in the range of 17 to 28 MPa^{0.5}; and

(iii) a particulate filler selected from MQ resin, silica, mica, titanium dioxide, zinc oxide, calcium carbonate or clays.

It is preferred that the composition additionally comprises a plasticizer.

According to another aspect of the present invention there is provided a method of reducing perspiration comprising the step of applying a composition as claimed in the first aspect on to the desired skin surface.

### DETAILED DESCRIPTION OF THE INVENTION

These and other aspects, features and advantages will become apparent to those of ordinary skill in the art from a reading of the following detailed description and the appended claims. For the avoidance of doubt, any feature of one aspect of the present invention may be utilized in any other aspect of the invention. The word “comprising” is intended to mean “including” but not necessarily “consisting of” or “composed of.” In other words, the listed steps or options need not be exhaustive. It is noted that the examples given in the description below are intended to clarify the invention and are not intended to limit the invention to those examples per se. Similarly, all percentages are weight/weight percentages unless otherwise indicated. Except in the operating and comparative examples, or where otherwise explicitly indicated, all numbers in this description and claims indicating amounts of material or conditions of reaction, physical properties of materials and/or use are to be understood as modified by the word “about”. Numerical ranges expressed in the format “from x to y” are understood to include x and y. When for a specific feature multiple preferred ranges are described in the format “from x to y”, it is understood that all ranges combining the different endpoints are also contemplated.

The compositions of the invention are typically “personal care compositions”, suitable for cosmetic use as detailed below. Further, use of the compositions of the invention is typically cosmetic, non-therapeutic use.

In some embodiments of the present invention, the compositions may be used for the therapeutic treatment of hyperhidrosis (extreme sweating).

By “An Antiperspirant Composition” as used herein, is meant to include a composition for topical application to the skin of mammals, especially humans. Such a composition is preferably of the leave-on type. By a leave-on composition is meant a composition that is applied to the desired skin surface and left on for a period of time (say from one minute to 24 hours) after which it may be wiped or rinsed off with water, usually during the regular course of personal washing. The composition may also be formulated into a product which is applied to a human body for improving the appearance, cleansing, odor control or general aesthetics. The composition of the present invention can be an anhydrous composition which is preferred to be delivered from an aerosol/propellant containing composition. Alternately it may also be delivered in the form of a gel or in stick form or may be delivered through a roll-on device. It is especially useful for delivering compositions to the axilla of an individual for anti-perspirancy benefits. “Skin” as used herein is meant to include skin on any part of the body where one may sweat (e.g., neck, chest, back, arms, underarms, hands, legs, buttocks and scalp) especially the underarms.

The present invention is directed to delivering a film forming polymer selected from modified cellulose having a solubility in water of less than 1 wt %; an organic solvent having a Hansen Solubility Parameter and in the range of 18 to 28 MPa^{0.5}; and a particulate filler selected from MQ resin, silica, mica, titanium dioxide, zinc oxide, calcium carbonate or clays.

It is preferred that the Hansen solubility parameter of the organic solvent is close to that of the film forming polymer. It is further preferred that the organic solvent is a mixture of two solvents.

The film forming polymer is a modified cellulose. The preferred modified cellulose is an alkyl cellulose. Preferred alkyl cellulose is one having 1 to 7 preferably 1 to 4 carbon atoms in the alkyl chain. Thus, the preferred film forming polymers are C1 to C7, more preferably C1 to C4 alkyl cellulose. The most preferred alkyl cellulose is ethyl cellulose. Ethyl cellulose is available as Ethocel (Dow chemicals) and as Aqualon (Ashland).

It is preferred that the ethyl cellulose for use in the invention has a degree of substitution (DS) in the range of 1.7 to 2.8, preferably in the range of 1.8 to 2.4.

It is preferred that the film forming polymer is included in 1.5 to 25%, preferably 2.5 to 20%, further more preferably from 5 to 15% by weight of the composition.

It is preferred that the composition comprises an organic solvent having a Hansen Solubility Parameter in the range of 17 to 28 MPa^{1/2}, preferably in the range of 18 to 25 MPa^{1/2}.

Hansen solubility is used to determine the degree of compatibility and miscibility of two materials. For example, we can judge the solubility and compatibility of a solvent and polymer to form a homogeneous mixture. The Hansen solubility parameter HSP is the sum of the δD for Dispersion (van der Waals), δP for Polarity (related to dipole moment) and δH for hydrogen bonding forces. The closer the HSP value of two materials, the higher is the compatibility of the two materials to blend with each other in solution. The Hansen partial solubility parameters of the organic solvent preferred for use in the present invention are in the ranges: non-polar (δd) from 16.6 to 17.3, polar (δp) from 6.6 to 8.3, and hydrogen bond (δh) from 8.5 to 10.0.

Examples of suitable organic solvents that could be used are ethanol, toluene, glycol ethers, or isododecane, preferably ethanol and glycol ethers. most preferably ethanol. Dowanol is a glycol ether which is another preferred solvent for use in the present invention. In a preferred aspect, when the comprises two solvents, they are preferably ethanol and glycol ether. The preferred glycol ether is Dowanol.

Dowanol is a glycol ether. It is the tradename for the compound with the chemical formula Propylene Glycol n-Propyl Ether C3H7OCH2CH(CH3)OH. The chemical structure is as given below:

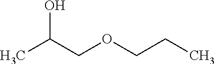

It is preferred that the solvent is included in 40 to 97%, preferably 75 to 90% by weight of the composition. It is further preferred that the film forming polymer is included in 1.5 to 25% by weight of the solvent.

The composition comprises a particulate filler selected from MQ resin, clay mica, titanium dioxide, zinc oxide, calcium carbonate or silica, preferably MQ resin, silica or clay, most preferably MQ resin. MQ resin is a branched, cage like oligo-silicone. It represents a broad range of hydrolytic condensation products of monofunctional silane (M) and tetrafunctional silane (Q), were synthesized by reaction of water glass with hexamethyldisiloxane. For all MQ copolymers, their high-molecular and middle fractions are solid substances, and the low-molecular fraction was a liquid. Glass transition temperature of high molecular mass fractions are either above the decomposition temperature or above 300° C., while the average molecular mass of other fractions has glass transition temperature in the range of 80 to 200° C., For liquid resins, with low molecular mass Tg is in the range of −40° C. to −10° C. They have good solubility both in organic solvents and in polydimethylsiloxanes (PDMS). MQ resin is also a good reinforcing additive for films.

It is preferred that the particulate filler is included in 1.5 to 20%, preferably 2.5 to 15% by weight of the composition. Further, it is preferred that the total amount of film forming polymer and particulate filler is not more than 25% by weight of the composition.

The composition preferably comprises a plasticizer.

Plasticizer as per this invention is a material that is included in the composition to reduce the brittleness and increase the flexibility of the film formed as a consequence of applying the composition on a desirable substrate e.g. the skin. In other words, they increase the elasticity of the film and make them softer.

Preferred plasticizer for use in the present invention is selected from one or more of polypropylene oxide, PPO in short (having a glass transition temperature T_{g} of less than 25° C.), cyclohexyl ethyl phthalate, cyclohexyl methyl phthalate, tricresyl phosphate, dibutyl phthalate, triethyl phosphate, diethyl phthalate, tripehenyl phosphate, diisopropyl phthalate, dimethyl phthalate, dioctyl phthalate, mineral oils, refined acetyl tributyl citrate, castor oil, acetyl triethyl citrate, corn oil, butyl stearate, cottonseed oil, dibutyl sebacate, dibutyl tartrate, diisobutyl adipate, glycerol Monostearate, oleic acid, stearic acid, methoxyethyl oleate, butoxyethyl stearate, polyethylene glycols (like PEG 4000 or PEG 6000), triethyl citrate, cetyl alcohol, tributyl citrate, myristyl alcohol, tributyrin (glycerol tributyrate), or stearyl alcohol.

Of the above, the more preferred plasticizers for use in the present invention are one or more of triethyl citrate, polypropylene oxide (with Tg lower than 25° C.), cyclohexyl ethyl phthalate, dibutyl phthalate, triethyl phosphate, diethyl phthalate, mineral oils, butyl stearate, cottonseed oil, dibutyl sebacate, dibutyl tartrate, diisobutyl adipate, glycerol monostearate, stearic acid, polyethylene glycols (like PEG 4000 or PEG 6000), cetyl alcohol, tributyl citrate, myristyl alcohol, tributyrin (glycerol tributyrate), or stearyl alcohol. The most preferred plasticizer is selected from triethyl citrate, PPO (with Tg lower than 25° C.), glycerol monostearate, stearic acid, cetyl alcohol, triethyl phosphate, mineral oil.

An especially preferred plasticizer is polypropylene oxide (PPO) which is also known as poly propylene glycol (PPG). PPO for use in the present invention preferably has a glass transition temperature (Tg) of less than 25° C. The glass transition temperature of a polymer is the temperature at which polymers change from a hard and rigid state to a more flexible and supple state. Tg occurs in a temperature range over which the mobility of the polymer chains increases significantly. The right plasticizer alters the property of the polymer film render it the desired flexibility and opacity. Polypropylene glycol is a polymer derived from monomer propylene glycol. The term polypropylene glycol or PPG is used for low to medium molecular weight polymer when the nature of the end-group, which is usually a hydroxyl group gives significant contribution to the overall property of the polymer. The PPG for use in the present invention is preferably selected from PPG P1200 or PPG P2000 which are available from Dow chemicals

Without wishing to be bound by theory, the inventors believe that the polymers like ethyl cellulose together with the particulate filler forms a uniform occlusive film over the sweat ducts after application and thereby reduce perspiration. Ethyl cellulose in combination with the right solvent or mixture of solvents forms a robust and adherent film on the skin even in presence of sweat. The strength and other sensory characteristics of the film are enhanced by the inclusion of the plasticizer.

The very specific combination of the film forming polymer, the particulate filler and the solvent or solvent mixture are then decided by utilizing the principle that the Ra value as calculated below has a value as low as possible preferably in the range of 5 to 7.

In the calculation of Ra, the various Hansen solubility parameters of the various ingredients or mixtures are taken and the Ra value calculated as below:

For a Mixture:

δD total=x₁δD₁+(1−x₁)δD₂

Similarly, δP total and δH total are calculated.

Ra is calculated using the formula below:

4(δD₁−δD₂)²+(δP₁−δP₂)²+(δH₁−δH₂)²=Ra²

In the above equation 1 denotes the solvent blend and 2 denotes the mixture of the film forming polymer (e.g. ethyl cellulose) and particulate filler (e.g. MQ resin) blend.

Values of the various parameters for the various commonly used ingredients are given below:

| | δD | δP | δH |
|---|---|---|---|
| Ethanol | 15.8 | 8.8 | 19.4 |
| Dowanol | 15.1 | 5.7 | 11.7 |
| Ethyl cellulose | 17 | 7.7 | 9.6 |
| Silica/MQ resin | 18.5 | 7.5 | 9 |

In a highly preferred aspect, the composition comprises a mixture of Ethanol and Dowanol. When both solvents are included, the weight ratio of Dowanol to Ethanol is in the range of 1:1 to 1:9. In a further highly preferred aspect, the composition comprises ethyl cellulose as the film forming polymer and MQ resin as the particulate filler. The weight ratio of ethyl cellulose to MQ resin is preferably in the range of 1:9 to 9:1. Under these highly preferred conditions the Ra value is in the range of 5 to 7 which the present inventors have determined to ensure that the composition exhibits the desired antiperspirancy benefits with acceptable to good sensorial properties.

Antiperspirant compositions of the present invention may advantageously comprise an additional, non-film forming polymer based antiperspirant active. Whilst this might be a conventional antiperspirant salt comprising Al and/or Zr, such as aluminium chlorohydrate or aluminium-zirconium chlorohydrate optionally complexed with glycine, it is preferred that any additional antiperspirant active is not of this type.

The anti-perspirant active can be applied cosmetically and topically to the skin. In the method of the invention, the composition is wiped across the surface of the skin, depositing a fraction of the composition as it passes. The composition of the invention may be delivered through a stick, in a gel form, using a roll on or through a spray/aerosol. The preferred form of delivering the present invention is a spray/aerosol.

### Aerosol Compositions

The antiperspirant composition may be delivered through an aerosol composition which may comprise a propellant in addition to the other ingredients described hereinabove.

Commonly, the propellant is employed in a weight ratio to the base formulation of from 95:5 to 5:95. Depending on the propellant, in such aerosol compositions the ratio of propellant to base formulation is normally at least 20:80, generally at least 30:70, particularly at least 40:60, and in many formulations, the weight ratio is from 90:10 to 50:50. A ratio range of from 70:30 to 90:10 is sometimes preferred.

Propellants herein generally are one of three classes; i) low boiling point gasses liquifided by compression, ii) volatile ethers and iii) compressed non-oxidising gases.

Class i) is conveniently a low boiling point material, typically boiling below −5° C., and often below −15° C., and in particular, alkanes and/or halogenated hydrocarbons. This class of propellant is usually liquefied at the pressure in the aerosol canister and evaporates to generate the pressure to expel the composition out of the canister. Examples of suitable alkanes include particularly propane, butane or isobutane. The second class of propellant comprises a very volatile ether of which the most widely employed ether hitherto is dimethyl ether. This propellant can advantageously be employed at relatively low weight ratio of propellant to base formulation, for example to as low as 5:95. It can also be employed in admixture with, for example, compressible/liquefiable alkane gasses. The third class of propellant comprises compressed non-oxidising gasses, and in particular carbon dioxide or nitrogen. Inert gases like neon are a theoretical alternative A suitable propellant for use in the present invention is diethanol amine.

When the composition of the invention is delivered in a roll-on, a firm solid or a stick format, the topically acceptable carrier comprises a hydrophobic carrier or an aqueous carrier. The hydrophobic carrier in such cases may comprise a silicone compound, low boiling alcohol or a wax. When the composition comprises a propellant it is delivered as an aerosol.

### Stick or Soft Solid Compositions

Many different materials have been proposed as gellant for a continuous oil phase, including waxes, small molecule gelling agents and polymers. They each have their advantages and of them, one of the most popular class of gellant has comprised waxes, partly at least due to their ready availability and ease of processing, including in particular linear fatty alcohol wax gellants. A gelled antiperspirant composition is applied topically to skin by wiping it across and in contact with the skin, thereby depositing on the skin a thin film.

The nature of the film depends to a significant extent on the gellant that is employed. Although wax fatty alcohols have been employed as gellant for many years, and are effective for the purpose of gelling, the resultant product is rather ineffective at improving the visual appearance of skin, and in particular underarm skin, to which the composition has been applied. This problem has been solved by including ameliorating materials for example, di or polyhydric humectants and/or a triglyceride oil.

### Roll-On

Liquid compositions that are applicable from a roll-on broadly speaking can be divided into two classes, namely those in which an antiperspirant active is suspended in a hydrophobic carrier, such as a volatile silicone and those in which the antiperspirant active is dissolved in a carrier liquid. The latter has proven to be more popular. There are mainly two sorts of dissolving carrier liquid, namely carriers that are predominantly alcoholic, which is to say the greater part of the dissolving carrier fluid comprises ethanol and the second class in which the carrier liquid is mainly water. The former was very popular because ethanol is a mild bactericide in its own right, but its popularity waned because it stings, especially if the surface onto which the composition has been applied has been damaged or cut, such as can easily arise during shaving or other de-hairing operations.

The second class of formulations that is an alternative to alcoholic formulations comprise a dispersion of water-insoluble or very poorly water soluble ingredients in an aqueous solution of the antiperspirant. Herein, such compositions will be called emulsions. Antiperspirant roll-on emulsions commonly comprise one or more emulsifiers to maintain a distribution of the water-soluble ingredients.

The composition of the present invention can comprise a wide range of other optional components. The CTFA Personal care Ingredient Handbook, Second Edition, 1992, which is incorporated by reference herein in its entirety, describes a wide variety of non-limiting personal care and pharmaceutical ingredients commonly used in the skin care industry, which are suitable for use in the compositions of the present invention. Examples include: antioxidants, binders, biological additives, buffering agents, colorants, thickeners, polymers, astringents, fragrance, conditioners, exfoliating agents, pH adjusters, preservatives, natural extracts, essential oils, skin sensates, skin soothing agents, and skin healing agents.

The present invention also provides for a method of reducing perspiration comprising the step of applying the composition of the first aspect on to the desired skin surface. The skin surface could be any topical surface which is prone to sweating especially the axilla i.e. the underarm portion of the human body. The method is preferably non-therapeutic.

The invention will now be demonstrated with the help of the following non-limiting examples.

### EXAMPLES

The following compositions as shown in Table-1 were prepared.

The compositions were tested for permeance of the membrane on which the composition is coated, as described below:

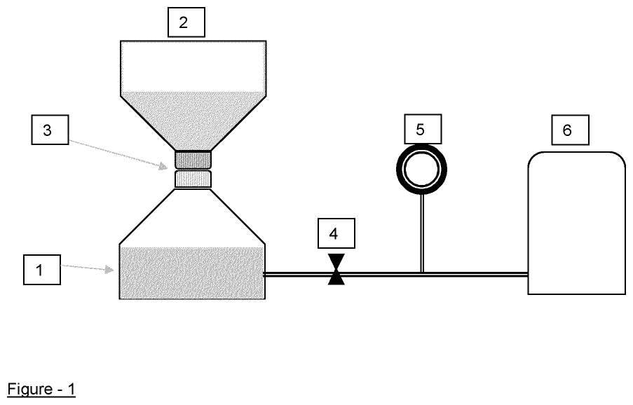

A Membrane Permeance Cell-Sweat-o-meter developed in house was used to carry out the experiment. A schematic of the apparatus is shown in FIG. 1.

As shown in FIG. 1, a membrane made of PCTE (polycarbonate membrane) of 10 micron pore size and 47 mm diameter procured as PCTF10047100 from Sterlitech was placed in between the membrane holder (3) separating the water reservoir (2) and collection chamber (1). The membrane is coated with the composition being tested. The water reservoir (2) is filled with water and a negative pressure is applied using the vacuum pump (6). The suction pressure is controlled using valve (4) and the suction pressure is measured using pressure gauge (5). At the desired suction pressure, the water from the reservoir passes through the membrane and is collected in the collection chamber (1) which is calibrated. The volume of water collected with time is noted.

70 g of distilled water is poured into the water reservoir. The valve is opened to the desired pressure. The experiment is carried out for a sufficient time to generate a mass-time profile over a set interval e.g. to record the flow across the membrane between 10 and 60 g (50 g total). The data on the permeance is summarized in Table-1.

The sensorial aspects of the composition on application to skin was measured as given below:

Sensory evaluation was done on visual basis considering parameters such as opacity of film formed after the solvent has evaporated, flexibility, tackiness, and defects/wrinkles formed.

A set of at least 5 respondents were asked to evaluate the films on above mentioned parameters on a scale of 1 to 5, with 5 being more acceptable and 1 the least. An ideal film should not be opaque, flexible enough to feel comfortable to skin, non-tacky, and homogeneous.

The data on the permeance (in terms of flow rate) and the sensory score are summarized in the Table below:

TABLE 1

| | | Flow rate | |
|---|---|---|---|
| Example | Composition | (g/s) | Sensory |
| A | 5% Ethyl cellulose + rest Ethanol | 2.28 | 1 |
| 1 | 4% Ethyl cellulose + 1% MQ100 + | 2.77 | 2 |
| | rest Ethanol | | |
| 2 | 4% Ethyl cellulose + 1% MQ100 + | 1.68 | 4 |
| | 75% Ethanol and 20% Dowanol | | |

It is preferred that the desirable composition has a permeance (low flow rate) of less than 3 g/s, preferably less than 2 g/s, more preferably less than 1.5 g/s. It is also preferred that the sensory score is as high as possible. The data in the Table-1 above indicates that the compositions as per the invention, Examples 1 and 2, provide for the desired permeance. With use of two solvents (Example 2), as compared to one solvent (Example 1) not only does the permeance increase but the sensory is also considerably better.

1. An anti-perspirant composition comprising:
(i) a film forming polymer selected from modified cellulose having a solubility in water of less than 1 wt %;
(ii) an organic solvent having a Hansen Solubility Parameter in the range of 17 to 28 MPa^{0.5}; and
(iii) a particulate filler selected from MQ resin, silica, clays, or combinations thereof,
wherein the modified cellulose is a C1 to C7 alkyl cellulose.

2. The composition as claimed in claim 1, wherein the modified cellulose is a C1 to C4 alkyl cellulose.

3. The composition as claimed in claim 16 wherein the ethyl cellulose has a degree of substitution (DS) in the range of 1.7 to 2.8.

4. The composition as claimed in claim 1, wherein Hansen partial solubility parameters of the organic solvent are in the ranges: non-polar (δd) from 16.6 to 17.3, polar (δp) from 6.6 to 8.3, and hydrogen bond (δh) from 8.5 to 10.0.

5. The composition as claimed in claim 1, wherein the organic solvent is selected from ethanol, toluene, glycol ethers, isododecane, or combinations thereof.

6. The composition as claimed in claim 5, wherein the organic solvent is a mixture of ethanol and glycol ether.

7. The composition as claimed in claim 1, wherein the film forming polymer is included in the composition at an amount of 1.5% to 25% by weight of the composition.

8. The composition as claimed in claim 1, wherein the organic solvent is included in the composition at an amount of 50% to 97% by weight of the composition.

9. The composition as claimed in claim 1, wherein the particulate filler is included in the composition at an amount of 1.5% to 20% by weight of the composition.

10. The composition as claimed in claim 1, further comprising a plasticizer.

11. The composition as claimed in claim 17, wherein the polypropylene oxide is selected from PPG P1200. PPG P2000, or combinations thereof.

12. The composition as claimed in claim 10, wherein the plasticizer is included in the composition at an amount of 1.5% to 10% by weight of the composition.

13. The composition as claimed in claim 10, wherein the total amount of the film forming polymer and the plasticizer is not more than 25% by weight of the composition.

14. A method of reducing perspiration comprising the step of applying the composition as claimed in claim 1 on to a desired skin surface.

15. The composition as claimed in claim 1, wherein the particulate filler is MQ resin.

16. The composition as claimed in claim 1, wherein the modified cellulose is ethyl cellulose.

17. The composition as claimed in claim 10, wherein the plasticizer is polypropylene oxide with glass transition temperature less than 25° C.
